(19) United States
(12) Patent Application Publication
(10) Pub. No.: US 2011/0071086 A1
(43) Pub. Date: Mar. 24, 2011

(54) COMPOSITIONS FOR TISSUE REPAIR/REGENERATION

(76) Inventors: Victor J. Dzau, Durham, NC (US); Jian Guo, Durham, NC (US)

(21) Appl. No.: 12/741,112

(22) PCT Filed: Nov. 4, 2008
(86) PCT No.: PCT/US2008/012442

§ 371 Date: Nov. 30, 2010

Related U.S. Application Data
(60) Provisional application No. 61/001,890, filed on Nov. 4, 2007.

(51) Int. Cl. A61K 38/17; C12N 5/071; C07K 14/47; A61P 9/10; A61P 9/00

(52) U.S. Cl. 514/16.4; 435/375; 530/350

(57) ABSTRACT

The invention provides compositions and methods for cardiac repair and/or regeneration of tissues such as myocardium.

### RELATED APPLICATIONS

This application claims priority to U.S. provisional application No. 61/001,890, filed on Nov. 4, 2007, the entire contents of which is hereby incorporated by reference.

### GOVERNMENT SUPPORT

This invention was made with Government support under grants HL72010, HL73219, HL58516, and HL35610 awarded by the National Institutes of Health. The Government has certain rights in the invention.

### FIELD OF THE INVENTION

The present invention relates to tissue regeneration and repair.

### BACKGROUND

Each year millions of Americans experience acute myocardial infarction (AMI); a significant portion (19%) die from loss of functional cardiac tissue. Congestive heart failure is a major and largely irreversible problem for those who survive. Stem cell-based therapies offer a potential means of regenerating damaged or dead myocardium. While early attempts at delivering cells into infarcted tissue have demonstrated modest improvements in cardiac function, such approaches are associated with potentially undesirable side effects.

### SUMMARY OF THE INVENTION

Paracrine factors secreted by Akt-modified stem cells have been shown to protect cardiomyocytes from, e.g., after, ischemic injury. The invention provides a method for reducing cell death or regenerating injured tissue by contacting an injured or diseased tissue with a composition comprising a purified Hypoxia regulated Akt mesenchymal Stem cell (MSC) Factor (HASF). For example, the tissue is cardiac tissue such as the myocardium. The composition comprises the amino acid sequence of SEQ ID NO:1 or 2. The cardiac muscle has been damaged by disease, such as a myocardial infarction. By regenerating an injured myocardial tissue is meant restoring ventricular function and/or decreasing infarct size. Ventricular function is measured by methods known in the art such as radionuclide angiography.

A method for reducing myocardial infarct size is carried out by administering to an individual suffering from or having suffered from a myocardial infarction, a composition comprising purified HASF. Optionally, the method includes a second therapeutic agent such as an anti-apoptotic agent, a protein kinase C (PKC) modulator, or an anti-thrombotic agent.

The composition is administered to the subject prior to, at the time of, or shortly after (1, 5, 10, 15, 30, 60 minutes; 1.5, 2, 4, 6, 12, 18, 24, 48 hours) identification of cell damage or identification of a symptom of ischemia or reperfusion injury. For example the composition is administered to a subject prior to a cardiac event or ischemic-reperfusion injury. Such a subject is a risk candidate for an ischemic event or condition. Symptoms of a cardiac event include for example, chest pain, arm pain, fatigue and shortness of breath. For example, the composition is administered at the onset of symptoms, e.g., chest pain, associated with a cardiac event such as a myocardial infarction. The composition is administered systemically or locally. For example, the composition is administered directly, i.e., by myocardial injection to the cardiac tissue, or systemically, e.g., interperitoneally, orally, intravenously. In another example, administration of the composition is carried out by infusion into a coronary artery. Slow-release formulations, e.g., a dermal patch, in which diffusion of the composition from an excipient such as a polymeric carrier mediates drug delivery are also within the invention. Optionally, the subject is further administered VEGF or thyrosin beta 4.

The composition is administered at a dose sufficient to inhibit apoptotic death or oxidative stress-induced cell death of myocardial tissue. To determine whether the composition inhibits oxidative-stress induced cell death, the composition is tested by incubating the composition with a primary or immortalized cell such as a cardiomyocyte. A state of oxidative stress of the cells is induced (e.g., by incubating cells with H₂O₂), and cell viability is measured using standard methods. As a control, the cells are incubated in the absence of the composition and then a state of oxidative stress is induced. A decrease in cell death (or an increase in the number of viable cells) in the compound treated sample indicates that the composition inhibits oxidative-stress induced cell death. Alternatively, an increase in cell death (or an decrease in the number of viable cells) in the compound treated sample indicates that the composition does not inhibit oxidative-stress induced cell death. The test is repeated using different doses of the composition to determine the dose range in which the composition functions to inhibit oxidative-stress induced cell death.

A subject to be treated is suffering from or at risk of developing a condition characterized by aberrant cell damage such as oxidative-stress induced cell death (e.g., apoptotic cell death) or an ischemic or reperfusion related injury. A subject suffering from or at risk of developing such a condition is identified by the detection of a known risk factor, e.g., gender, age, high blood pressure, obesity, diabetes, prior history of smoking, stress, genetic or familial predisposition, attributed to the particular disorder, or previous cardiac event such as myocardial infarction or stroke.

Conditions characterized by aberrant cell damage or death include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia, ischemic hepatitis, hepatic vein thrombosis, cirrhosis, portal vein thrombosis, pancreatitis, ischemic colitis, or myocardial hypertrophy as well as brain disorders such as autism. Cardiac repair or regeneration is evaluated by detecting an improvement of symptoms such as chest pain or shortness of breath as well as by evaluation of heart function by standard methods such as cardiac magnetic resonance, echocardiography, and/or ventricular angiography.

Also within the invention is a cell culture or preservation media containing purified HASF and a method of maintaining inhibiting stem cell differentiation, e.g., inhibiting myogenesis, by contacting a population of isolated stem cells with purified HASF. Isolated stem cells are selected from the group consisting of embryonic stem cells, mesenchymal stem cells, and hematopoetic stem cells. Stem cells are isolated from the tissue of origin by fractionation by cell surface markers or other distinguishing characteristics. Preferably, a population of isolated cells is at least 85% stem cells. More preferably, the population is 90, 95, 98, 99, 100% stem cells. HASF is also useful to induce adult cardiomyocytes to re-enter the cell cycle and thereby contribute to tissue regeneration and repair. Preservation of cells in this manner is useful in transport and storage of stem cells prior to transplantation into a subject for therapeutic purposes

The compositions described herein are purified, e.g., synthetically produced, recombinantly produced, and/or biochemically purified. A purified composition such as a protein or peptide is at least 60%, by weight, free from proteins and naturally occurring organic molecules with which it is naturally associated. Preferably, the preparation is at least 75%, more preferably 90%, and most preferably at least 99%, by weight, the desired composition. A purified antibody may be obtained, for example, by affinity chromatography. By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

By “substantially identical,” when referring to a protein or polypeptide, is meant a protein or polypeptide exhibiting at least 75%, but preferably 85%, more preferably 90%, most preferably 95%, or even 99% identity to a reference amino acid sequence. For proteins or polypeptides, the length of comparison sequences will generally be at least 20 amino acids, preferably at least 30 amino acids, more preferably at least 40 amino acids, and most preferably 50 amino acids or the full length protein or polypeptide. Nucleic acids that encode such “substantially identical” proteins or polypeptides constitute an example of “substantially identical” nucleic acids; it is recognized that the nucleic acids include any sequence, due to the degeneracy of the genetic code, that encodes those proteins or polypeptides. In addition, a “substantially identical” nucleic acid sequence also includes a polynucleotide that hybridizes to a reference nucleic acid molecule under high stringency conditions.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference.

The term “isolated DNA” is meant DNA that is free of the genes which, in the naturally occurring genome of the organism from which the given DNA is derived, flank the DNA. Thus, the term “isolated DNA” encompasses, for example, cDNA, cloned genomic DNA, and synthetic DNA.

As is well known in the medical arts, dosage for any one animal depends on many factors, including the animal's size, body surface area, age, the particular compound to be administered, sex, time and route of administration, general health, and other drugs being administered concurrently. Subjects to be treated include humans, companion animals such as dogs, cats as well as horses, oxen, donkey, cow, sheep, pig, rabbit, monkey or mouse.

The invention also includes the use of HASF in the manufacture of a medicament to reduce cell death in an ischemic tissue as well as the use of HASF in the manufacture of a medicament for preserving or storing a tissue or organ ex vivo.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, GenBank/NCBI accession numbers, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A is a bar graph showing apoptosis in myocytes. The open reading frame of stem cell secreted paracrine factor (HASF, 1158 bp) was amplified by standard PCR and cloned in-frame with maltose binding protein (MBP) to generate MBP-HASF fusion proteins. This MBP-HASF fusion protein was purified by standard affinity chromatography and further by FPLC. H9C2 myocytes were incubated first with PBS control, MBP control and MBP-HASF at different concentrations for 30 min, and then challenged with 100 μM of H₂O₂ for 2 h. Early apoptosis was analyzed by staining cells with Annexin V/Propidium Iodine (PI) and quantified by flow cytometer. Y axis represents the % of the sum of Annexin V positive cells+Annexin V/PI double positive cells. Compared with PBS control and MBP control, this novel MBP-HASF fusion protein at the concentration of 1 nM and 10 nM had modest protection against H₂O₂ induced early apoptosis around 16% and 30% respectively. * indicates statistical significance of P<0.05, and ** means P<0.01. Values are means±SD in triplicates.

FIG. 1B is a photograph of a Northern blot assay. Tissue specific expression of mouse HASF. A PCR fragment (626 bp) of mouse HASF was amplified by PCR and radio labeled with 32P as the probe for northern blotting. There are prominent bands (˜4 kb) indicating the expression of mouse HASF in ovary, brain, liver and embryo, with a modest expression in the lung, thymus, spleen and heart, and no expression in the kidney and testes.

FIG. 1C(a) is a bar graph showing Affymetrix microarray expression data of mouse HASF in Akt-MSCs and Control-MSCs under hypoxia (H) and/or normoixa (N) conditions for 6 h. Y axis represents the relative expression level in microarray gene chip. HASF was dramatically up-regulated (˜4 fold increase) in Akt-MSCs especially under hypoxic condition. *** indicates statistical significance of P<0.001. Values are means±SD in triplicates.

FIG. 1C(b) is a photograph of an electrophoretic gel. RT-PCR validation of mouse HASF expression in Akt-MSCs and control MSCs under hypoxia (H) and/or normoxia (N) conditions for 6 h. A PCR fragment (626 bp) of mouse HASF was amplified by standard PCR. HASF was preferentially up-regulated in Akt-MSCs under hypoxic conditions, consistent with Affymetrix microarray expression. Mouse beta actin was used as the internal control. Lane 1, Akt-MSCs under normoxia; lane 2, Akt-MSCs under hypoxia; lane 3, control MSCs under normoxia; lane 4, control MSCs under hypoxia.

FIG. 1D is a photograph of an electrophoretic gel. The full-length cDNA of human HASF without the stop codon was amplified by standard PCR and cloned in the Gateway Entry vector first for sequencing and then recombined in Destination vector 40 as V5 epitope tagged HASF at the carboxyl terminus. HEK 293 cells were transiently transfected with/without this expression construct. The supernatants from transfected cells were separated on 10% SDS-PAGE gel and probed with a rabbit anti-V5 antibody for western blotting. Human HASF protein tagged with V5 epitope was detected as ˜40 KDa protein bands in the supernatant of transfected HEK 293 cells, but not in the supernatant of control lipofectamine transfected cells. Lane 1, control lipofectamine transfected cells; lane 2 and 3, with HASF expression construct, 24 h and 48 h after transfection respectively.

FIG. 1E(a., b.) are photographs of electrophoretic gels. a.) Coomassie staining of the expression of HASF recombinant protein. The opening reading frame of human HASF (1158 bp) was amplified by PCR and cloned in-frame in pET 15b vector to generate 6×His tagged HASF recombinant protein. HASF protein was cysteine-rich and expressed exclusively as a ˜40 KDa protein in the ‘inclusion bodies’ 3 h after induction of 1 mM of IPTG at 28° C. Lane 1, protein marker; lane 2, before induction; lane 3, 3 h after induction; lane 4, insoluble fraction as inclusion bodies; lane 5, soluble fraction. b.). Comassie staining of recombinant 6×His tagged HASF protein after purification and refolding. Lane 1, protein marker; lane 2-7, increasing amount of 6×His tagged HASF recombinant protein from 50 ng up to 500 ng after refolding.

FIG. 2A is a bar graph showing apoptosis in myocytes. H9C2 myocytes were pre-incubated ±10 nM of HASF recombinant protein for 30 min, and then challenged with 100 μM of H₂O₂ for 2 h. Apoptosis was quantified on a flow cytometer with Annexin V and Propidium Iodine (PI) staining. Y axis means the % of the sum of Annexin V positive cells+Annexin V/PI double positive cells. Compared with the vehicle control PBS treated cells, pre-incubation of H9C2 myocytes with 10 nM of the HASF recombinant protein significantly (˜50%) reduced H₂O₂ induced apoptosis after refolding as the 6×His tagged HASF protein. Human recombinant IGF protein was used as a positive control. ** indicate statistical significance of P<0.01 and *** for P<0.001. Values are means±SD in triplicates.

FIG. 2B is a two-panel bar graph showing caspase activity. Adult rat cardiomyocytes were freshly isolated and treated ±10 nM of HASF recombinant protein for 30 min, and then challenged with 100 μM of H₂O₂ for various time points. H₂O₂ induced apoptosis in cardiomyocytes was evidenced by the dynamic increase in the activities of the initiator Caspase 9 and effector Caspase 3/7 at 5 h, 7 h and 9 h respectively. Y axis is the relative amount of luminescence indicating the relative amount of active Caspase activities. Pre-incubation of the cardiomyocytes with 10 nM of HASF recombinant protein significantly inhibited the activities of both Caspase 9 and Caspase 3/7 at different time points, about ˜36% reduction of Caspase 9 and ˜42% reduction of Caspase 3/7 respectively. *** indicate statistical significance of P<0.001. Values are means±SD in triplicates.

FIG. 2C is a photograph of an electrophoretic gel. Adult rat cardiomyocytes were treated with ±10 nM of HASF recombinant protein for 30 min, and then challenged with 100 μM of H₂O₂ for overnight (˜15 h). Genomic DNA was extracted and separated on 1% agarose gel. H₂O₂ induced apoptosis in cardiomyocytes was companied by typical DNA fragmentation (laddering) at late-stage apoptosis. Pre-incubation of cardiomyocytes with 10 nM of HASF recombinant protein inhibited the DNA laddering. Lane 1, DNA marker; lane 2, −H₂O₂ control; lane 3-4, +H₂O₂; lane 5-6, +HASF recombinant protein and then +H₂O₂.

FIG. 2D is photograph of an electrophoretic gel. Adult rat cardiomyocytes were treated with ±10 nM of HASF recombinant protein for 30 min, and then challenged with 100 μM of H₂O₂ for 6 h. Mitochondrial fraction or cytosolic fraction or total cell lysate were extracted and separated in 15% SDS-PAGE and transferred to nitrocellulose membrane and probed with mouse anti-Cytochrome C monoclonal antibody. During H₂O₂ induced apoptosis in cardiomyocytes, there was a slight decrease of mitochondrial Cytochrome C where it usually abundantly resides but with a marked release of it into cytosolic fraction. Pre-incubation of cardiomyocytes with HASF recombinant protein for 30 min significantly inhibited this Cytochrome C release from mitochondria into cytosol. Lane 1-2, −H₂O₂ control; lane 3-4, +H₂O₂ control; lane 5-8, four individual samples of +HASF recombinant protein and then +H₂O₂.

FIG. 2E is a photograph of an electrophoretic gel. During H₂O₂ induced apoptosis in cardiomyocytes, there was a modest lost of Bcl-2 in the mitochondrial fraction and marked translocation of Bax from cytosol onto mitochondria. However, pre-incubation of cardiomyocytes with HASF recombinant protein for 30 min maintained and slightly increase Bcl-2 protein level on the mitochondria but does not influence the translocation of Bax protein from cytosolic compartment onto mitochondria. Lane 1, −H₂O₂ control; lane 2, +H₂O₂ control; lane 3-6, four individual samples of +HASF recombinant protein and then +H₂O₂.

FIG. 3A (left panel) is a photograph of heart tissue. FIG. 3A (right panel is a bar graph showing infarct size. Rats were randomly divided into PBS control group and HASF recombinant protein injected group, n=10 for each group. The reperfusion injury model includes a 30 min coronary ligation with the immediate injection of vehicle PBS control or 1 μg of HASF recombinant protein into 5 locations in a total volume of 250 μl in the myocardium below ligation suture, followed by loosening the ligation suture for another 24 h to achieve reperfusion injury. Area at risk (AAR) was calculated as the left ventricular total area excluding Evans Blue dye positive area, and % infarct area was calculated as the % of infarct area/AAR. The mean of % of infarct area for all sections of each heart was calculated blindly for comparisons using ImageJ computer software. Three representative photos from each group were shown. *** indicates statistical significance of P<0.001. The 30 min ischemia and followed by 24 h reperfusion resulted in 36.4±8.4% of myocardium infarction. However, intramyocardium injection of 1 μg of HASF recombinant protein during the initial 30 min ischemia period significantly reduced the infarction down to 15.3±3.4%, with a dramatic ˜58% reduction in the infarct size observed.

FIG. 3B (left panel) is a photograph of cardiomyocytes; FIG. 3B (right panel is a bar graph showing apoptosis. Other groups of rats were used for TUNEL staining to detect in vivo apoptosis of cardiomyocytes during 30 min/24 h reperfusion. Serial cryosections of 5 μm thick were made immediately below the ligation area, 10 sections for each heart were analyzed, with 8 rats in each group. Sections were also counterstained with hematoxylin. Negative control was carried out with the same procedure except for adding rTdT enzyme. Total number of dark-brown color stained apoptotic nuclei were counted and added up blindly in 10 randomly taken fields within the peri-infarct region in each group. *** indicates statistical significance of P<0.001. HASF recombinant protein injection significantly reduced in vivo cardiomyocyte apoptosis by 69%.

FIG. 3C (left panel) is a photmicrograph heart tissue sections; FIG. 3C (right panel is a bar graph showing fibrosis. For fibrosis analysis, animals were sacrificed 4 weeks after the initial 30 min ischemia/24 h reperfusion injury and serial cyrosections of 5 μm thick were made immediately below the ligation area, 10 sections for each heart analyzed, and with 8 rats in HASF protein injected group and 6 rats in PBS injected control group. Brilliant blue color stained collagen area was quantified using ImageJ computer software and the mean of % fibrosis was calculated as collagen positive area/total area. PBS injected control animals displayed extensive collagen deposition (14.6±2.6%) after initial reperfusion injury followed by the 4 weeks remodeling period; meanwhile, injection of 1 μg of this HASF recombinant protein during the 30 min ischemia period resulted in only 5.7±1.5% of collagen deposition, with a 61% reduction of fibrosis observed. *** indicates statistical significance of P<0.001.

FIG. 4A is a photograph of an electrophoretic gel. Adult rat cardiomyocytes were freshly isolated and incubated with 10 nM of HASF recombinant protein for various time points. Cells lysates were separated on 10% SDS-PAGE gel and probed with different phosphor-specific antibodies for PI3K-Akt family. Incubation of cardiomyocytes with HASF recombinant protein significantly phosphorylated AktThr308, peaking at 30 min which decreased slightly afterwards and then increased up again at 3 h time point. No marked phosphorylation of AktSer473 could be detected and adding HASF did not change the level of total Akt protein.

FIG. 4B is a photograph of an electrophoretic gel. Adult rat cardiomyocytes were first treated ±10 μM of PI3K-Akt inhibitor LY 2940002 for 3 h and then incubated with 10 nM of HASF recombinant protein for another 30 min. This transient phosphorylation and activation of AktThr308 in cardiomyocytes was almost completely abolished by pre-incubation PI3K inhibitor. Lane 1, −inhibitor, −HASF; lane 2, +inhibitor, −HASF; lane 3-5, −inhibitor, +HASF; lane 6-8, +inhibitor, +HASF.

FIG. 4C is a photograph of an electrophoretic gel. Further analysis of Akt downstream target genes revealed a coincident phosphorylation of GSK3βSer9 at 30 min and 3 h, and a gradually increased phosphorylation of proapoptotic BadSer128 at 2-3 h. No effect could be observed on the PI3K negative regulator-PTENSer380.

FIG. 5A. and FIG. 5B are dot plots showing the results of enzymatic assays with synthetic peptide Akt Thr308 (FIG. 5A) or Akt Ser473 (FIG. 5B). Primarily cultured rat adult cardiomyocytes were stimulated with 10 nM of HASF or vehicle control PBS for 10 min. Cells were lyzed and fractionated by HPLC into ˜60 fractions (100 μl) on Mono Q ion-exchange column according to proteins' charge. An aliquot of 10 μl of each fraction was assayed with the addition of 10 μl of 150 μM of ATP-³²P and 200 μM of Akt Thr308 peptide for 15 min at 30° C. Reactions were quenched by adding 20 μl of 3% H₂SO₄ and 10 μl of the total 40 μl reaction mixture was spotted onto P81 paper. After five times washing with 3% H₂SO₄, the incorporation of ³²P onto the peptide was quantified and expressed as counts per minute (cpm) with a liquid scintillating counter. There is a distinct radioactivity peak (fraction 13-17) with Akt Thr308 peptide in HASF stimulated cardiomyocytes compared with vehicle control PBS, FIG. 5A. No peaks could be observed with Akt Ser473 peptide either in the control or HASF stimulated groups, FIG. 5B.

FIG. 5C is a photograph of an electrophoretic gel. Fractions from 5-20 with Akt Thr308 peptide assay in HASF stimulated group were separated by SDS-PAGE gel and silver stained. Visible protein bands were cut off and subjected to mass-spectrometry sequencing analysis to identify the potential kinase (s) that phosphorylates Akt Thr308 peptide. Cyclin H, the regulator for CDK7, was found in fractions 13-16 which was indicated with arrows.

FIG. 5D is a photograph of an electrophoretic gel. Fractions 11-18 were further separated by SDS-PAGE gel and probed with antibody for CDK7. Although the absolute amount of CDK7 kinase was not visible in silver stained gel, the western blotting signal of CDK7 kinase started from fraction 12 and went up in fractions 13-14, peaking at fraction 15, and went down in fractions 16-17. The appearance of CDK7 was consistent with the radioactivity peak in the enzymatic assay with Akt Thr308 peptide.

FIG. 5E is a bar graph showing phosphorylation levels. Using the commercially available recombinant enzyme of CDK7/cyclin H/MAT1 triple complex (Upstate/Millipore Corp.), the similar enzymatic assay was repeated with Akt Thr308 peptide. This pure recombinant enzyme demonstrated a dramatic phosphorylation of Akt Thr308 peptide, in a dose-dependent manner, reaching almost 10,000 cpm with only 50 ng and above 20,000 cpm with 500 ng of recombinant enzyme.

FIG. 5F is a bar graph showing phosphorylation level. To avoiding potential artifact with short peptide assays, a further validation was performed using the unactive full-length Akt protein, which was activated by CDK7/cyclin H/MAT1 triplex complex enzyme. As shown in. FIG. 5F, Akt substrate peptide-Akt/SGK peptide, was strongly phosphorylated by the full-length unactive Akt protein which was activated by CDK7/cyclin H/MAT1 enzyme, in the similar dose-dependent manner as well. There was no 32P incorporation onto Akt/SGK peptide in the two negative controls, either without the enzyme, or without full-length unactive Akt protein.

FIG. 6 is a an alignment showing the relationship of mouse and human HASF.

### DETAILED DESCRIPTION

Purified HASF is useful in the treatment of a variety or disorders characterized by aberrant cell damage or death due to ischemia or other insults. Such conditions include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension; kidney disorders such as renal failure, kidney ischemia; liver disorders such as ischemic hepatitis, hepatic vein thrombosis, cirrhosis, portal vein thrombosis; pancreatitis; ischemic colitis; or myocardial hypertrophy. HASF is also useful for treatment of brain disorders such as autism in which the gene has been found to be mutated (Morrow et al., 2008, Science 321:218-223).

Cell death associated with tissue or organ grafts, e.g., liver graft, is reduced by contacting the tissue with purified HASF, e.g., as a component of a transport or organ storage solution to reduce cell injury/death during time outside of the body. An organ, e.g., kidney, heart, lung, or liver, to be transplanted is bathed in a solution containing HASF. For example, HASF is added to known tissue/organ preservation solutions such as Viaspan, a.k.a., University of Wisconsin (UW) solution (Potassium lactobionate: 100 mM; KH₂PO₄: 25 mM; MgSO₄: 5 mM; Raffinose: 30 mM; Adenosine: 5 mM; Glutathione: 3 mM; Allopurinol: 1 mM; and, Hydroxyethyl starch: 50 g/L) (Belzer, et al., U.S. Pat. No. 4,798,824, issued Jan. 17, 1989) or Histidine-Tryptophan-Ketoglutarate (HTK) solution (Pokorny et al., 2004, Transpl. Int. 17:256-260). Other tissue/organ preservation or storage solutions to which HASF is added include Stanford University solution (Swanson, D. K., et al., Journal of Heart Transplantation, (1988), vol. 7, No. 6, pages 456-467) and modified Collins solution (Maurer, E. J., et al., Transplantation Proceedings, (1990), vol. 22, No. 2, pages 548-550; Swanson, D. K., et al.).

HASF is administered alone or in combination with other agents such as antiapoptotic drugs (e.g., IDN-6556 for liver graft protection, Georgiev et al., 2007, Liver Transplantation 13:318-320); tauroursodeoxycholic acid for protection against neurological injury after stroke (Rodrigues et al., 2003, PNAS 100:6087-6092) or Protein Kinase C (PKC) modulators (e.g., modulators of PKC-delta such as KAI-9803 (Kai Pharmaceuticals, Inc.); Circulation, 2008; 117:886-896) for reduction of reperfusion injury and cell death or injury due to stroke or myocardial infarction. HASF is also administered together with other cell protective agents such as Sfrp-2. In a combination therapy approach, HASF boosts the efficacy of other therapeutic agents.

Purified HASF has been recombinantly produced, and tested in an animal ligation-reperfusion model of acute myocardial infarction. Administration of HASF as a therapeutic agent recapitulates the biological action of stem cells without stem cell-related drawbacks, a clear advantage for clinical use.

HASF is used for cell protection and reduction of tissue damage in emergency as well as elective settings. For example, in an emergency situation such as acute myocardial infarction (AMI), HASF is administered directly into the myocardium (direct myocardial injection), intravenously, or by intracoronary catheterization. HASF is also administered to reduce or prevent cell death in the context of stroke, e.g., in the case of hemorrhagic stroke, HASF administered systemically gains access to brain tissue (gray, white matter) due to disruption of the blood/brain barrier. The agent is also optionally administered directly to affected, e.g., flooded, brain tissue. For ischemic stroke, HASF may be injected into the carotid artery.

In an elective setting, HASF is administered at indications of angina or other cardiac or coronary disorders. For example, catheterization and administration of HASF is carried out on one day, followed by surgery days later, e.g., 1-30, 1-10, 1-5, 1-3, or 1-2 days post-catheterization/HASF administration. Other non-emergent situations include orthopedic surgery in which the main artery to the area of work is tied off, the surgical procedure is carried out, and then blood flow to the area of work is restored. Cell death damage is reduced in this situation by bathing the surgical area with a solution containing HASF or injecting HASF directly into the blood vessel.

HASF confers clinical benefit in any ischemic organ system, i.e., any organ or tissue that is subjected to a situation characterized by reduced blood flow. For example, tissue is preserved and cell death is prevented or reduced in hypoxic areas associated with peripheral artery disease, critical limb ischemia, or arterial emboli. In the former cases, HASF is directly injected into the affected site. In the latter case, HASF is infused after removal of the embolus or emboli. Similarly, HASF is administered to ocular tissue in the case of retinal artery occlusion as a one time dose or to treat recurrent emboli.

### Identification and Characterization of HASF

Akt-stem cells produce paracrine factors upon exposure to hypoxic conditions. For example, HASF is produced by the cell and stored in the Golgi apparatus, and exposure of the cell to a hypoxia triggers secretion.

An Akt-regulated stem cell paracrine factor was found to protects ischemic hearts through the Activation of Cyclin-Dependent Kinase 7 that selectively phosphorylates Akt308Thr. Mesenchymal stem cells (MSC) overexpressing Akt improve myocardial cell survival, repair and regeneration through the expression and release of paracrine factors that influence the microenvironment of the injured tissue. Disclosed herein is an Akt regulated stem cell factor that is upregulated in response to hypoxia. Using microarray expression profiling, 5 novel genes were identified that putatively encoding secreted proteins that are differentially expressed in Akt MSC. These genes were cloned and expressed in E coli and screened for biological activities using initially a H₂O₂ induced apoptosis assay of H9C2 cardiomyocytes in vitro. This gene was upregulated by hypoxia and exerts a cytoprotective effect on H9C2 cells. The gene was cloned into pEt15b vector to allow rapid purification as a 6×His tagged recombinant protein. This Hypoxia regulated Akt MSC Paracrine Factor (HASF) meets the criteria of a biologically relevant mediator: 1) it is differentially expressed in Akt cells in response to hypoxia; 2) it exerts cytoprotective effect on adult cardiomyoctes subject to hypoxic and oxidative injury; 3) when administered to animals with acute MI in vivo, it resulted in reduction in tissue injury and enhanced repair. HASF is a cysteine rich protein whose expression and secretion is inhibited by PI3K inhibitor LY294002. When added to cardiomyocytes, it activates PI3 kinase and results in downstream phosporylation of Akt that is independent of PDK1. Further analysis show that HASF activates cyclin H dependent kinase (CDK7) that uniquely phosphorylated Akt at Thr 308 and not Ser 473. This is associated with phosphorylation of GSK 3B Ser 28 and Bad Ser 9, inhibition of the release of mitochondrial cytochrome C, maintenance of mitochondrial Bcl2 and reduction in Caspase 9 and Caspase 3/7 activities, Annexin staining as well as DNA laddering.

HASF increases target cell survival through a unique CDK that activates Akt selectively via Thr 308 phosporylation. The unique signaling effects of HASF underscore the critical role of Akt in regulating and mediating cell survival and tissue repair.

### Paracrine Mediators

The maintenance, expansion and proliferation of stem cells are highly dependent on the stem cell microenvironment. Much has been studied on the biology of the stem cell niche. Stem cells themselves express and secrete autocrine/paracrine mediators that support the microenvironment. These mediators exert autocrine effects on stem cell biology including survival, self renewal and growth. Stem cells secrete proteins that participate in the effects of stem cells in tissue repair and regeneration. This paracrine mechanism is supported by the demonstration that many cytokines with cytoprotective and growth properties are released by the stem cells in areas of tissue injury. Stem cells contribute to tissue repair and regeneration by releasing a variety of factors in a dynamic spatiatemporal manner that can lead to cell survival, angiogenesis, tissue repair and remodeling, as well as cellular regeneration.

Hypoxia induced phosporylation and activation of Akt that resulted in increased MSC viability and engraftment in vivo. Akt MSC transplantation into ischemic hearts led to dramatic reduction in tissue injury and to remarkable cardiac repair and restoration of ventricular function. A significant part of these effects is explained by the release of paracrine mediator(s) by these MSC. This paracrine mechanism of stem cell action on tissue repair and regeneration is supported by observations of the release of cytokines by stem cells and identification of the upregulation of 50 or more secreted proteins in the Akt MSC. These results highlight several important biologic process in stem cell biology and action for tissue protection and repair: 1) the expression and release of paracrine factors contribute significantly to stem cell action, 2) Akt is a critical signal molecule responsible for cell survival and function, and 3) hypoxia is an important stimulus for the activation of Akt and its physiologic consequence.

Physiologic genomics was used to further identify novel molecules that are involved in this pathway, especially those which are Akt regulated that subsequently contribute to cell survival and tissue repair. A hypoxia induced Akt regulated gene (HASF) whose product activates a novel enzyme that phosporylates Akt was identified and characterized. HASF plays an essential role in MSC autoregulation by Akt resulting in increased stem cell survival and engraftment, as well as in the MSC paracrine action that increases cardiomyocyte survival and tissue repair.

HASF is expressed and secreted by MSC that overexpress Akt. This factor which is regulated by Akt increases cell survival through the activation of CDK7 that phosphorylates Akt selectively at Thr 308. HASF is a critical molecule released by stem cells that mediates Akt autocrine actions and exerts stem cell paracrine effects on tissue protection and repair. HASF is useful in the treatment of tissue injury (e.g., myocardial ischemia) for repair and regeneration.

### Therapeutic Methods

The methods of inhibiting cell or tissue damage and ischemic or reperfusion related injuries are carried out by contacting myocardial tissue with purified HASF. Also included are methods of regenerating injured myocardial tissue. The therapeutic methods include administering to a subject, or contacting a cell or tissue directly with a composition containing a purified cytoprotective compound such as HASF or another purified Akt-MSC paracrine factor. Cell/tissue damage is characterized by a loss of one or more cellular functions characteristic of the cell type which can lead to eventual cell death. For example, cell damage to a cardiomyocyte results in the loss contractile function of the cell resulting in a loss of ventricular function of the heart tissue. An ischemic or reperfusion related injury results in tissue necrosis and scar formation. An increase in contractile function, improvement of ventricular function, as well as a reduction in tissue necrosis or scar formation occurs after administration of HASF.

Injured myocardial tissue is defined for example by necrosis, scarring or yellow softening of the myocardial tissue. Injured myocardial tissue leads to one or more of several mechanical complications of the heart, such as ventricular dysfunction, decrease forward cardiac output, as well as inflammation of the lining around the heart (i.e., pericarditis). Accordingly, regenerating injured myocardial tissue results in histological and functional restoration of the tissue. The cell is any cell subject to apoptotic or oxidative stress induced cell death. For example, the cell is a cardiac cell such as a cardiomyocyte, a liver cell or a kidney cell. Tissues to be treated include a cardiac tissue, a pulmonary tissue, or a hepatic tissue. For example, the tissue is an muscle tissue such as heart muscle. The tissue has been damaged by disease or deprivation of oxygen.

Cells or tissues are directly contacted with HASF, e.g. by direct injection into the myocardium. Alternatively, HASF is administered systemically, e.g., infused into a blood vessel such as intracoronary artery. HASF is administered in an amount sufficient to decrease (e.g., inhibit) apoptosis induced or oxidative stress induced cell death as compared to untreated cells or tissues. Cells undergoing apoptosis are identified by detecting cell shrinkage, membrane blebbing, caspase activation, chromatin condensation and fragmentation as is well know in the art. Cell undergoing oxidative stress are identified by detecting an increase production of reactive oxygen species (ROS). A decrease in cell death (i.e., an increase in cell viability) is measured by using standard cell viability measurements such as BrdU incorporation assay and trypan blue exclusion.

The methods are useful to alleviate the symptoms of a variety disorders, such as disorders associated with aberrant cell damage, ischemic disorders, and reperfusion related disorders. For example, the methods are useful in alleviating a symptom of stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy. The disorders are diagnosed and or monitored, typically by a physician using standard methodologies. Alleviation of one or more symptoms of the disorder indicates that the compound confers a clinical benefit, such as a reduction in one or more of the following symptoms: shortness of breath, fluid retention, headaches, dizzy spells, chest pain, left shoulder or arm pain, and ventricular dysfunction

### Therapeutic Administration

The invention includes administering to a subject a composition comprising HASF. An effective amount of a therapeutic compound administered systemically in the range of about 0.1 mg/kg to about 150 mg/kg. Proteins or peptides are administered directly into the heart by injection at a dose of 1-1000 μg. For example, 10, 20, 30, 40, 50, 60, 75, 100 μg are administered by myocardial injection. Purified HASF is also administered by intracoronary delivery (e.g., via catheter) at a dose of 0.1-10 mg. For example, 2 mg of HASF is infused into an intracoronary artery after detection of myocardial infarction to minimize myocardial damage.

Effective doses vary, as recognized by those skilled in the art, depending on route of administration, excipient usage, and coadministration with other therapeutic treatments including use of other anti-apoptotic agents or therapeutic agents for treating, preventing or alleviating a symptom of a particular cardiac disorder. A therapeutic regimen is carried out by identifying a mammal, e.g., a human patient suffering from (or at risk of developing) an cardiac disorder, using standard methods.

The pharmaceutical compound is administered to such an individual using methods known in the art. Preferably, the compound is administered orally, nasally, topically or parenterally, e.g., subcutaneously, intraperitoneally, intramuscularly, and intravenously. The compound is administered prophylactically, or after the detection of an cardiac event such as a heart attack. The compound is optionally formulated as a component of a cocktail of therapeutic drugs to treat cardiac disorders. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for administration utilizing conventional methods. For example, HASF is formulated in a capsule or a tablet for oral administration. Capsules may contain any standard pharmaceutically acceptable materials such as gelatin or cellulose. Tablets are formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

HASF is effective upon direct contact with the affected tissue, e.g. heart muscle. Additionally, HASF is administered by implanting (either directly into an organ such as the heart or subcutaneously) a solid or resorbable matrix which slowly releases the compound into adjacent and surrounding tissues of the subject. For example, the composition is delivered to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen. Any variety of coronary catheter, or a perfusion catheter, is used to administer the compound. Alternatively, the compound is coated or impregnated on a stent that is placed in a coronary vessel.

For administration to the neurological tissues such as the brain, HASF is administered intravenously or intrathecally (i.e., by direct infusion into the cerebrospinal fluid). For local administration, a compound-impregnated wafer or resorbable sponge is placed in direct contact with CNS tissue. A biodegradable polymer implant such as a GLIADEL™ wafer is placed at the desired site. A biodegradable polymer such as a polyanhydride matrix, e.g., a copolymer of poly (carboxy phenoxy propane):sebacic acid in a 20:80 molar ratio, is mixed with a therapeutic agent, e.g., HASF and shaped into a desired form. Alternatively, an aqueous solution or microsphere formulation of the agent is sprayed onto the surface of the wafer prior to implantation. The compound or mixture of compounds is slowly released in vivo by diffusion of the drug from the wafer and erosion of the polymer matrix. Alternatively, the compound is infused into the brain or cerebrospinal fluid using known methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (e.g., an assembly described in U.S. Pat. No. 5,954,687) provides a fluid flow path suitable for the transfer of fluids to or from selected location at, near or within the brain to allow administration of the drug over a period of time.

The following materials and methods were used to generate the data described herein.

### Bioinformatics and Molecular Biology

GeneChip Mouse Genome 430A 2.0 Array (Affymetrix, Inc.) was used to discover differentially expressed novel transcripts in mouse Akt-MSCs. Novel transcripts and the predicted protein sequences from Akt-MSCs were assessed for being secreted proteins by the prediction of possessing a N-signal peptide (http://www.cbs.dtu.dk/services/SignalP/) and the exclusion of transmembrane domains (http://www.cbs.dtu.dk/services/TMHMM-2.0/). Potential biological function for novel proteins was predicted by online server (http://www.cbs.dtu.dk/services/ProtFun/). A PCR fragment (626 bp) of mouse HASF (Genebank accession no. NM_{—}001033145, with gene name as 1190002N15Rik), was amplified from mouse Akt-MSCs with the forward primer, 5′-ggccatttgcaaaatatcttggagcttgtg-3′ and reverse primer, 5′-acttaactgtgccagatagccacgcagtt-3′. This PCR product was subsequently cloned into pGEM-TA vector (Promega) for sequencing and was on the other hand, labeled with ³²P isotope as the probe for northern blotting (Ambion, FirstChoice Mouse blot 1). Human homologous cDNA of HASF, with gene name as chromosome 3 open reading frame 58, (C3orf58, Genebank accession no. BC037293) was purchased from American Type Culture Collection (ATCC, clone MGC 33365 or IMAGE 5267770). Full-length human cDNA of HASF without the stop condon was amplified by PCR and cloned in Gateway Entry vector for sequencing and subsequently recombined into Gateway destination vector 40 (Invitrogen) as the mammalian expression construct to generate the V5-epitope tagged HASF for transfection and detection in the culture medium of HEK293 cells by western blotting with rabbit anti V5 antibody (Abcam).

### Recombinant Protein Purification, Refolding and Mass Spectrometry

The open reading frame of human HASF without the predicted N-signal sequence (1158 bp) was cloned in-frame in pMal-2C vector (New England Biolabs) to generate a fusion protein of maltose binding protein MBP-HASF. The expression was induced by 0.3 mM of Isopropyl β-D-1-thiogalactopyranoside (IPTG) in E. coli. TB1 strain and purification of MBP-HASF was done by standard affinity chromatography according to New England Biolabs instructions and was further purified by FPLC system. The same open reading frame of human HASF (1158 bp) without N-signal sequence was next amplified with the forward primer (underlined with Nde I restriction site), 5′-ggcggccatatggaccggcgcttcctgcag-3′ and the reverse primer (underlined with BamH I restriction site), 5′-ggcggcggatccctacctcacgttgttacttaattgtgctagg-3′, which was cloned in-frame into pET 15b vector (EMD Biosciences) to generate 6×His tagged HASF recombinant proteins. The expression of this 6×His-HASF recombinant protein was induced for 3 h at 28° C. by adding 1 mM of IPTG in E. coli. BL21 (DE3) strain when the OD₆₀₀ reached 0.6 and expressed exclusively in inclusion bodies, which after washing and re-centrifuging extensively in large volume of 20 mM of Tris (pH 7.5), 10 mM of EDTA and 1% Triton X-100 for 6 times, protein pellet was subsequently solublized in a denaturing buffer containing 50 mM CAPS (pH11.0) and 0.2% of N-lauroylsarcosin, and refolded by extensive dialysis in 20 mM of Tris (pH 8.0) and 20 mM of NaCl with step-wise decreasing amount of dithiothreitol starting at 200 μM at 4° C. Promotion of intramoleculer disulfide bonds was further enhanced by adding a redox pair of 0.2 mM of oxidized v.s.1 mM of reduced glutathione at room temperature. Misfolded recombinant proteins were then precipitated and removed by centrifugation for 30 min at 4° C. Soluble recombinant proteins were further enriched through TALON affinity chromatography (Clontech) and after elution with 1 M of imidazole, pH 7.0, this 6×His-HASF recombinant protein was finally dialyzed at 4° C. overnight in a large volume of phosphate buffered saline (PBS), pH 7.4 and concentrated by centrifugation through the filtration tubes with 3 KDa molecular weight cut-off membranes (Sartorious/Vivascience) at 4° C. The 6×His-HASF recombinant proteins were then immediately stored at −80° C. in small aliquots and thawed only once for experiments. To confirm the protein sequences, the 6×His-HASF recombinant protein were subsequently digested with trypsin (0.6 μg), and the tryptic peptides were subjected to matrix-assisted laser desorption-ionization mass spectrometry (MALDI-MS) on an Applied Biosystems 4700 Proteomic Analyzer® time of flight (TOFTOF®) mass spectrometer. Positive mode time of flight was used to identify peptides, and individual peptides were sequenced by MS/MS using collision-induced dissociation. All sequence and peptide fingerprint data was searched using the SwissProt database and Mascot search engine.

### In Vitro Annex V/PI Staining, Caspase 3/7/9, DNA Fragmentation and Apoptosis-Related Genes Expression by Western Blotting

Rat myocytes-H9C2 cells were obtained from ATCC and cultured in DMEM medium containing 10% of FBS, supplemented with 2 mM of L-glutamine, 100 U/ml of penicillin and 100 μg/ml of streptomycin (Invitrogen). Cells were seeded one day before at 1×10⁵/well in 6-well plates. Recombinant protein HASF was added into cells the next day at the final concentration of 10 nM for 30 min, with same volume of PBS or same concentration of MBP used as controls, and then the cells were challenged with 100 μM of H₂O₂ for 2 h. Attached cells were trypsinized and combined with floating cells. H₂O₂ induced apoptosis was then analyzed on a flow cytometer for Annexin V/Propidium Iodine double staining with the Vybrant Apoptosis Assay Kit #2 (Invitrogen).

Adult rat ventricular cardiomyocytes were isolated from 6 weeks old female Sprague-Dawley rat (Harlan World Headquarters, Indianapolis, Ill., USA) hearts by enzymatic digestion as previously described⁸⁰ and were seeded in 6-well plates pre-coated with 1 μg/cm² of laminin (Sigma) at 5×10⁴/well and cultured overnight in serum-free M199 medium (Sigma), supplemented with 2 mM of L-carnitine, 5 mM of creatine, 5 mM of taurine, 0.2% of albumin, 100 U/ml of penicillin and 100 μg/ml of streptomycin. Recombinant protein HASF was added into cells the next day at the final concentration of 10 nM, with same volume of PBS used as vehicle controls, for 30 min, and then the cells were challenged with 100 μM of H₂O₂ for various time points. For Caspase assays, cardiomyocytes were scraped off plates in lysis buffer and were analyzed by a luminescent plate reader with Caspase-Glo 3/7 and Caspase-Glo 9 kits (Promega); and for DNA fragmentation, genomic DNA from cardiomyocytes was extracted and separated on 1% agarose gel electrophoreses, with Apoptotic DNA Ladder Extraction Kit (BioVision), according manufacturers' instructions. For western blotting of apoptosis-related gene expression, mitochondrial fraction or cytosolic fraction of cell lysate were first extracted respectively and separated in 15% SDS-PAGE and transferred to nitrocellulose membrane (Biorad), probed with mouse anti-Cytochrome C monoclonal antibody (Calbiochem), rabbit anti-Bcl-2 polyclonal antibody (Abcam), or rabbit anti-Bax polyclonal antibody (Abcam), and with rabbit anti-mouse or goat anti-rabbit secondary antibodies respectively (Abcam).

For cell signaling pathways and protein phosphorylation analysis, freshly isolated adult rat ventricular cardiomyocytes were seeded at 1×10⁵ in 6 cm laminin-coated dishes and cultured overnight in serum-free M199 medium with supplements mentioned above. PI3K inhibitor LY294002 or DMSO vehicle control was added next day at a final concentration of 10 μM for 3 h, prior to the addition of recombinant protein HASF at final concentration of 10 nM for various time points. Cells were lyzed in lysis buffer supplemented with both phosphatase and protease inhibitor cocktails (Sigma) and the total lysates were separated in 10% SDS-PAGE and transferred to Immun-Blot PVDF membrane (Biorad). Rabbit polyclonal antibodies for total Akt, phospho-Akt^{Ser473}, phospho-Akt^{Thr308}, phospho-PTEN^{Ser380}, phospho-GSK3β^{Ser9}, (Cell Signaling Technology) and phospho-Bad^{Ser128} (Abcam) were used as first antibodies and goat anti-rabbit antibody was used as secondary antibody for western blotting.

In Vivo model of Ischemia/Reperfusion Injury, Infarct Size, TUNEL and Fibrosis Assays

Female Sprague-Dawley rats were used for all in vivo experiments. A midsternal thoracotomy was performed to expose the anterior surface of the heart after anesthesia. The proximal left ascending coronary artery (LAD) was identified and a 6.0 suture (Ethicon) was placed around the artery and surrounding myocardium. Regional left ventricular ischemia was induced for 30 minutes by ligation of LAD, followed by immediate injection of 1 μg of recombinant protein HASF or PBS vehicle control in five spots of intramyocardium in a total volume of 250 μl. The ligature was loosened and reperfusion was achieved after 30 min of the ischemia period and the incision was closed and the animals were allowed to recover.

For infarct analysis, 24 h after reperfusion, the LAD was re-ligated and ˜300 μl of 1% Evans Blue in PBS (pH 7.4) was retrogradely infused into the heart in a 2-3 min period to delineate the nonischemic area. The heart was excised and rinsed in ice-cold PBS. Five biventricular sections of similar thickness were made perpendicular to the long axis of the heart and incubated in 1% triphenyl tetrazolium chloride (TTC, Sigma) in PBS (pH 7.4) for 15 minutes at 37° C. and photographed on both sides. Area at risk (AAR) was calculated as the left ventricular total area excluding Evans Blue dye positive area, and % infarct area was calculated as the % of infarct area/AAR. The mean of % of infarct area for all sections of each heart was calculated blindly for comparisons using ImageJ computer software, with 10 rats in each group.

For TUNEL staining (DeadEnd Colometric TUNEL System, Promega) after 30 min ischemia/24 h reperfusion, serial cyrosections of 5 μm thick were made immediately below the ligation area, 10 sections for each heart were analyzed, with 8 rats in each group. Briefly, cryosections were first fixed in cold methanol for 5 min, washed in PBS and treated with proteinase K for 30 min at room temperature. Biotinylated nucleotide mix and rTdT enzyme were added to catalyze the end-labeling reaction for 1 h at 37° C. Streptavidin-HRP and DAB chromogen components were added to allow colormetric development. Sections were also counterstained with hematoxylin. Negative control was carried out with the same procedure except for adding rTdT enzyme. Total number of dark-brown color stained apoptotic nuclei were counted and added up blindly in 10 randomly taken fields within the peri-infarct region in each group.

For fibrosis analysis, animals were sacrificed 4 weeks after the initial 30 min ischemia/24 h reperfusion injury and serial cyrosections of 5 μm thick were made immediately below the ligation area, 10 sections for each heart analyzed, and with 8 rats in HASF protein injected group and 6 rats in PBS injected control group. Collagen deposition within the infracted region was stained with Masson's Accustain Trichrome Stains (Sigma) according to manufacturer's instructions. Brilliant blue color stained collagen area was quantified using ImageJ computer software and the mean of % fibrosis was calculated as collagen positive area/total area.

### In Vitro Enzymatic Assays in Discovery of the Upstream Kinase Being Responsible for Akt^{Thr308} Phosphorylation

Two short peptides harboring either Thr308 or Ser473 of rat/human Akt protein were designed and synthesized by Genescript Corporation. Akt Thr308, RRRKDGATMKTFCGTPEYLAPEV and Akt Ser473, RRRVDSERRPHFPQFSYSASGTA. Three arginines were added in front for the affinity to P81 chromatography paper. Primarily cultured rat adult cardiomyocytes were stimulated by HASF at 10 nM final concentration for 10 min and cells were homogenized in a lysis buffer containing 25 mM of Tris-Cl, pH 7.5, 1 mM of DTT, 60 mM of MgCl₂, 0.2% of NP-40 and supplemented with protease/phosphatase inhibitors. Clarified supernatant were incubate with ATP-Sepharose beads to enrich bound kinases which were eluted by adding 100 mM ATP. Free ATP was then removed by a few times buffer exchange with a Centricon® Centrifugal Filter and the final enriched kinase/proteins were injected into HPLC and fractioned into ˜60 fractions with gradient NaCl by Mono Q ion exchange column (GE Healthcare). The content of each fraction were assayed for kinase activity by adding 150 μM of ATP, [γ-³²P] ATP (specific activity of ˜7500 cpm/pmol), and plus 200 μM of the synthetic peptide Thr308 or Ser473, at 30° C. for 15 min. The reaction was quenched with same volume of 3% H₃SO₄ (20 μl) and 10 μl aliquots from each reaction mixture was spotted on P81 chromatography paper, which were washed five times with 3% H₃SO₄ before measuring ³²P incorporation by a γ-scintillation counter. Interesting fractions containing radioactive peaks were separated on 10% SDS-PAGE and silver stained. Proteins bands were in gel-digested with trypsin (0.6 μg), and the tryptic peptides were subjected to nanospray electrospray ionization mass spectrometry (ESIMS) on an Applied Biosystems QSTAR® pulsar mass spectrometer and were sequenced by ESI-MS/MS using BioAnalyst software. CDK7/cyclin H/MAT1 recombinant kinase complex, Akt1/PKBα unactive recombinant protein and Akt/SGK substrate peptide were purchased from Upstate/Millipore Corp. CDK7 mouse monoclonal antibody was purchased from Cell Signaling Technology.

### Statistics

All the results are presented as the mean±SD or mean±SEM and were analyzed with unpaired student T test. Probability (P) values<0.05 were considered statistically significant.

### Bioinformatics, Tissue Distribution, Cloning, Expression of Stem Cell Paracrine Factor (BASF) and Making of its Recombinant Proteins

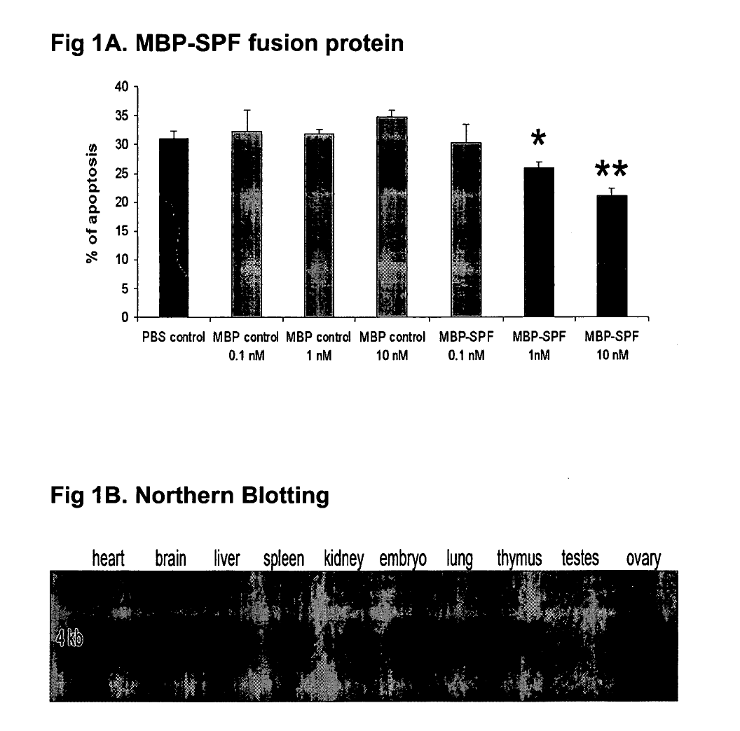

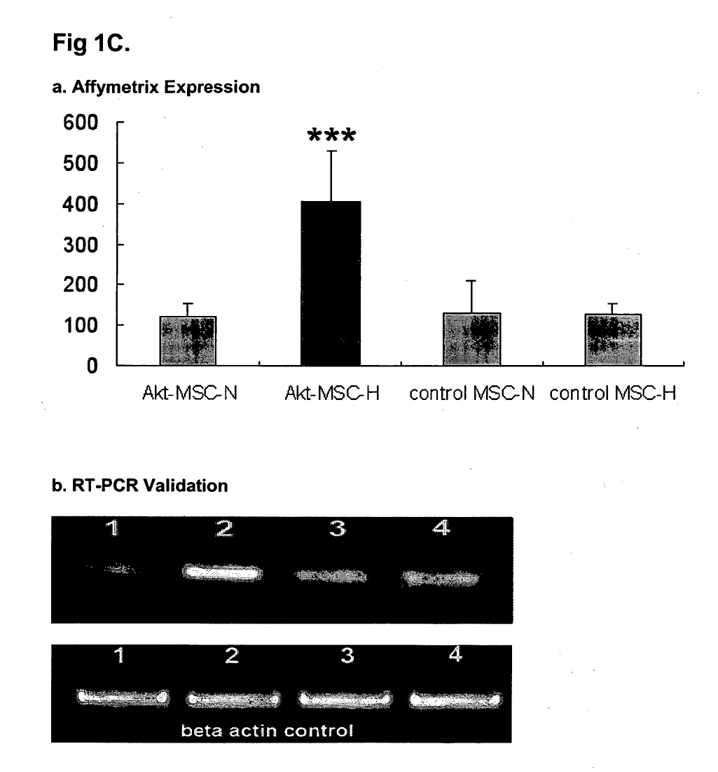

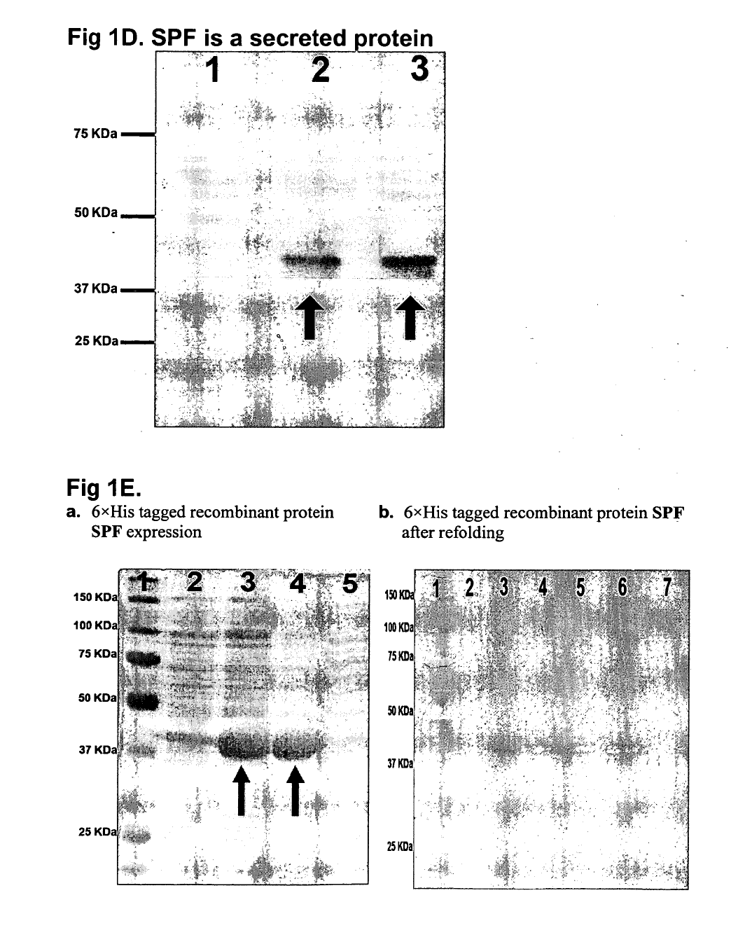

GeneChip Mouse Genome 430A 2.0 Array (Affymetrix, Inc.) was used to analyze the global expression of ˜14,000 mouse genes with over 22,600 probe sets in Akt-MSCs compared with control vector transduced MSCs under hypoxia or normoxia. In addition to some up-regulated secreted proteins with known paracrine function, e.g. pleiotrophin, chemokine ligands, some angiogenic and anti-apoptotic factors such as VEGF, IGF, bFGF, angiopoietin 4, HGF and etc, we further identified 11 novel transcripts that were differentially expressed in Akt-MSCs under hypoxia and/or normoxia. With the analysis of these 11 novels for being secreted proteins by the prediction of possessing the N-signal peptide (http://www.cbs.dtu.dk/services/SignalP/) and the exclusion of transmembrane domains (http://www.cbs.dtu.dk/services/TMHMM-2.0/), 5 novel transcripts potentially encoding secreted proteins were identified. The cDNA of these novel transcripts or their human homologues counterparts were subsequently cloned, expressed and purified as recombinant MBP-novel fusion proteins from E. coli. They were used in a pilot screening assay for being anti-apoptotic in H9C2 cells. One of these proteins which was named as HASF had a modest protection (˜30%) against H₂O₂ induced apoptosis (FIG. 1A), compared with vehicle control and MBP control. Gene ontology prediction of human HASF (Genebank accession no. BC037293, with gene name as C3orf58, chromosome 3 open reading frame 58) with the online server (http://www.cbs.dtu.dk/services/ProtFun/) indicated that HASF may function as a growth factor, with the first <45 amino acids as the N-signal peptide, without any O-/N-glycosylated sites and transmembrane domains predicted. There are 18 putative phosphorylation sites predicted at positions 13, 15, 38, 45, 114, 130, 157, 186, 189, 200, 223, 252, 332, 355, 373, 377, 378 and 387. The alignment with both human HASF and mouse HASF protein sequences (Genebank accession no. NM_{—}001033145, with gene name as 1190002N15Rik) revealed a highly conserved homology of about 98% similarities (FIG. 6).

A PCR fragment of 626 bp of mouse HASF was amplified from mouse Akt-MSCs under hypoxia and cloned into pGEM TA vector (Promega) for sequencing and the sequences were exactly identical corresponding to nucleotide position 885-1484 of mouse HASF (Genebank accession no. NM_{—}001033145). The same PCR fragment was also gel purified and labeled with 32P and used as the probe for tissue specified expression in northern blotting. As seen in FIG. 1B, mouse HASF mRNA (˜4 kb) was abundantly expressed in the ovary, brain, liver and embryo, with a modest expression in the lung, thymus, spleen and heart, and no expression in the kidney and testes could be observed. The same PCR fragment was amplified by reverse transcript PCR (RT-PCR) for the mouse HASF expression in Akt-MSCs and control MSCs under normoxia/hypoxia, and it was consistent with mouse HASF Affymetrix microarray expression data (FIG. 1C), in which mouse HASF is dramatically up-regulated in Akt-MSCs under hypoxic condition.

The full length cDNA of human HASF excluding the stop codon ‘TAG’ (1290 bp) was next amplified by standard PCR, cloned in-frame first in Gateway Entry vector for sequencing and subsequently recombined into Gateway destination vector 40 (Invitrogen) as the mammalian expression construct to generate a carboxyl-end-V5-epitope tagged HASF. Western blotting with rabbit anti V5 antibody confirmed the presence of V5-epitope tagged HASF in the culture media of HEK293 cells at 24 h and 48 h after transfection, but not in the medium of the vehicle control (lipofamtamine, Invitrogen) transfected HEK293 cells, indicating that HASF is a secreted protein (FIG. 1D).

Human HASF protein is rich in cysteine and contains 10 cysteine residues in this ˜40 KDa protein. Thus, the open reading frame of human HASF without N-signal region (1158 bp) was re-cloned into pET 15b vector to generate a 6×His-HASF recombinant protein, and as expected it was expressed exclusively in the inclusion bodies of E. coli. BL21 (DE3) strain. This 6×His-HASF recombinant protein was then solublized first in denaturing condition and refolded with step-wise decreasing amount of dithiothreitol and a redox pair to promote disulfide bond formation (FIG. 1E.). From 500 ml induced culture, approximately 1 mg of a total yield was obtained and at least 100 μg of this 6×His-HASF recombinant protein could be obtained after refolding. The protein sequence of this 6×His-HASF recombinant protein was further confirmed by mass spectrometry.

HASF Protected in vitro Cardiomyocyte Apoptosis

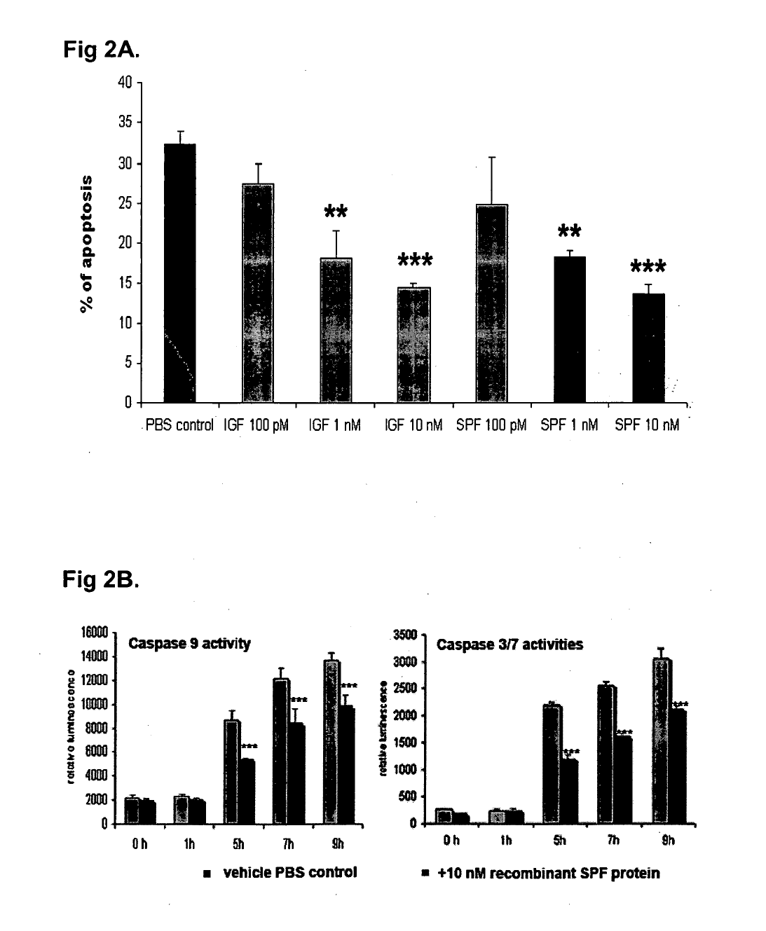

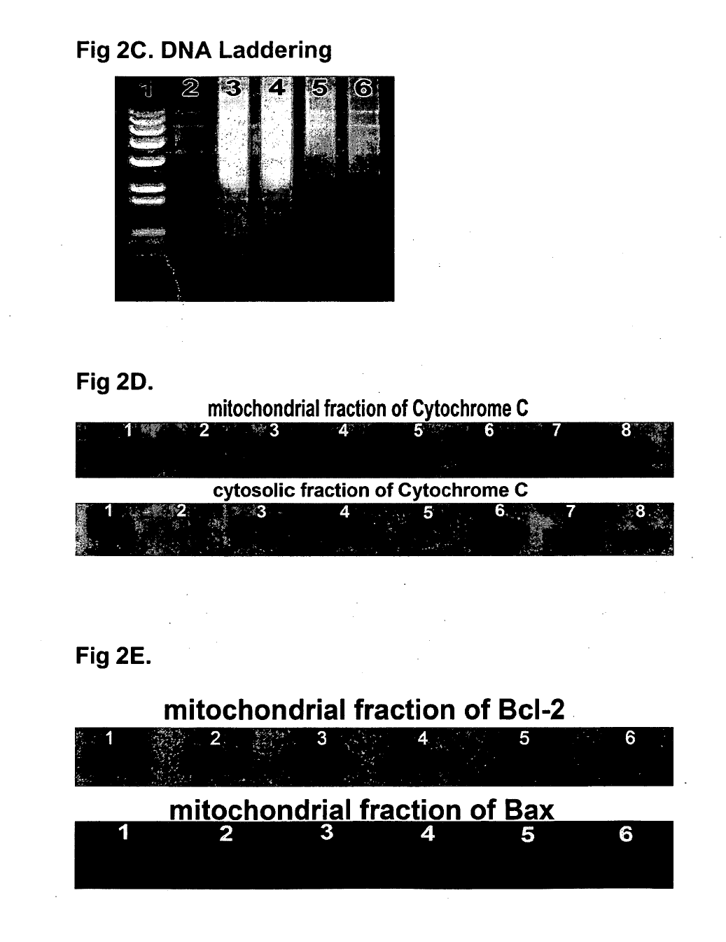

10 nM of HASF recombinant protein significantly (˜50%) reduced the H₂O₂ induced early apoptosis in H9C2 myocytes by Annexin V/PI staining, with a much stronger protection observed without the big MBP tag (˜42 KDa), see in FIG. 2A. This effect is comparable to 10 nM of human IGF recombinant protein (Invitrogen). Caspase 9 and Capase 3/7 assays were carried out in adult rat cardiomyocytes at various time points to observe the effect of HASF on the dynamic change of active Caspase activities. As shown in FIG. 2B, primary isolated and cultured adult rat cardiomyocytes displayed a dramatic increase of initiator Caspase 9 and the effector Caspase 3/7 after the challenge with 100 μM of H₂O₂, however, pre-incubation of 10 nM of HASF with cardiomyocytes for 30 min dramatically reduced both Caspase 9 and Caspase 3/7 at various time points, ˜36% reduction of Caspase 9 and ˜42% reduction of Caspase 3/7 respectively. Longer incubation about 15 h with 100 μM of H₂O₂ resulted in a typical DNA fragmentation/laddering, one of the hallmarks in late-stage apoptosis, in the genomic DNA of adult rat cardiomyocytes; nonetheless, pre-treatment of cardiomyocytes with 10 nM of HASF displayed a marked less DNA fragmentation (FIG. 2C). To investigate the effect of HASF on the apoptosis-related gene expression during H2O2 induced apoptosis, rat adult cardiomyocytes were pre-incubated with PBS vehicle control or 10 nM of HASF for 30 min, followed by stimulation by 100 μM of H₂O₂ for 6 h. As shown in FIG. 2D, the H₂O₂ induced apoptosis was accompanied by the dramatic release of Cytochrome C from mitochondria into cytosolic compartment, with a slight decrease of Cytochrome C in the mitochondrial fraction where it abundantly resides in the normal state. The pre-treatment of cardiomyocytes with HASF significantly prevented Cytochrome C release from mitochondria into cytosol. As seen in FIG. 2E, the H₂O₂ induced apoptosis is evidenced by a modest decrease of mitochondrial fraction of Bcl-2 and a marked translocation of Bax from cytosol onto mitochondria. The pre-treatment of cardiomyocytes with HASF slight increased and maintained the mitochondrial Bcl-2 protein level during apoptosis but did not prevent the translocation of Bax protein from cytosol onto mitochondria.

HASF Protected in vivo Cardiomyocyte Apoptosis, Reduced Infarct Size and Fibrosis

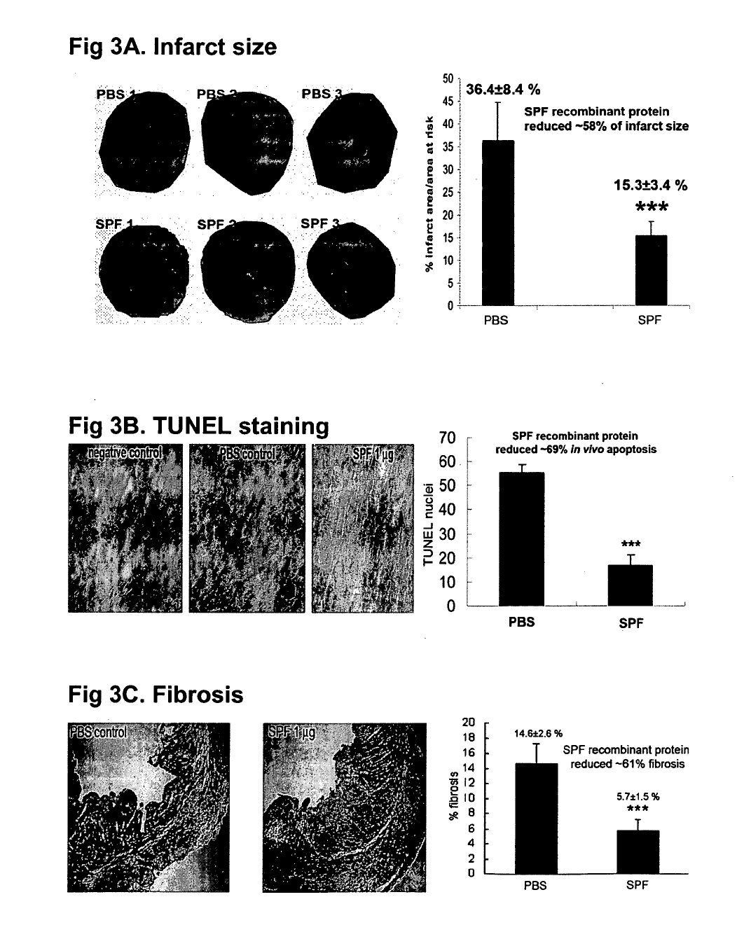

Experiments were carried out to determine whether the in vitro anti-apoptosis effect of HASF protected myocardium infarction in the animal model. As shown in FIG. 3A, 30 min ischemia and followed by 24 h reperfusion resulted in 36.4±8.4% of myocardium infarction evidenced by triphenyl tetrazolium chloride (TTC) and Evan's Blue stained cross sections in rat hearts. Intriguingly indeed, intramyocardium injection of 1 μg of this HASF recombinant protein during the initial 30 min ischemia period significantly reduced the infarction down to 15.3±3.4%, with a dramatic ˜58% reduction in the infarct size observed.

In FIG. 3B, 30 min ischemia/24 h reperfusion induced tremendous in vivo apoptotic cardiomyocytes within the peri-infarct region, demonstrated by dark-brown color stained nuclei with TUNEL method. Interestingly, a ˜69% reduction of the number of TUNEL positive nuclei was observed by injection of 1 μg of this HASF recombinant protein during the initial 30 min ischemia period, compared with the PBS control group.

To investigate the effect of HASF beyond the observed protection against acute apoptosis during reperfusion injury, we also analyzed the % of fibrosis which is indicated by collagen deposition with Masson's Accustain Trichrome staining, in the rats about 4 weeks later after the initial 30 min ischemia/24 h reperfusion. As seen FIG. 3C, PBS injected control animals displayed extensive collagen deposition (14.6±2.6%) after initial reperfusion injury followed by the 4 weeks remodeling period; meanwhile, injection of HASF recombinant protein during the initial 30 min ischemia period resulted in only 5.7±1.5% of collagen deposition, with a 61% reduction of fibrosis observed.

HASF Secreted from Akt-MSCs Activated Anti-Apoptosis PI3K-Akt Pathway in Rat Adult Cardiomyocytes through Paracrine Mechanism

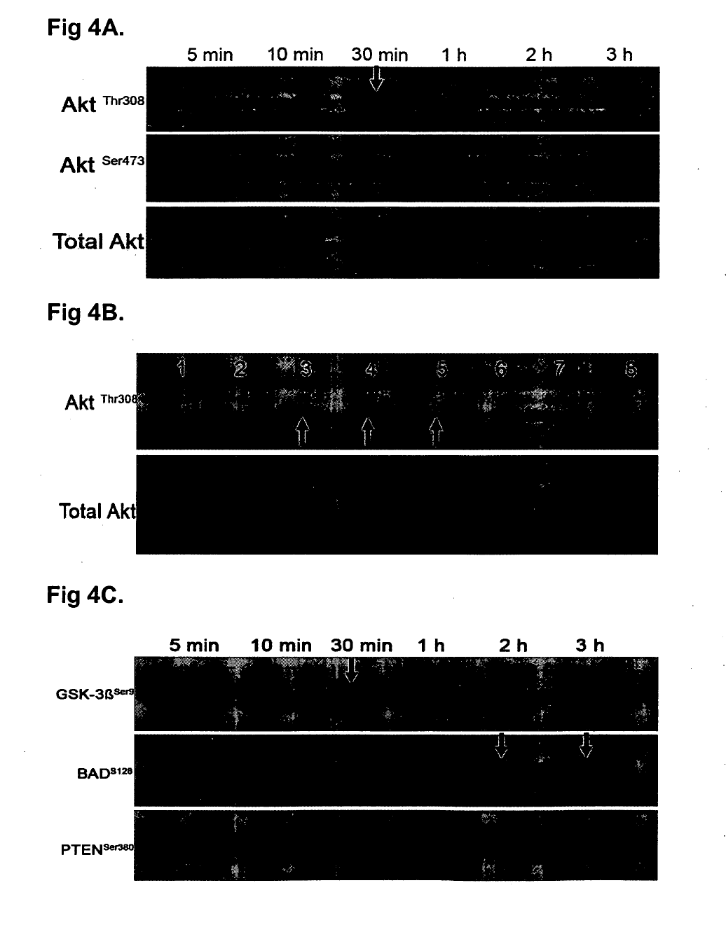

Since HASF was predicted as a growth factor and secreted from Akt-MSC especially under hypoxic condition, experiments were carried out to determine whether it could, in a paracrine fashion, deliver a survival signal via binding a cell surface receptor and/or receptor kinase, resulting in an intracellular activation of anti-apoptosis pathway(s) in the cardiomyocytes. Prominent phosphorylation of AktThr308 was observed peaking at 30 min in rat adult cardiomyocytes incubated with 10 nM of HASF, which decreased slightly afterwards and then increased up again at 3 h time point. No marked phosphorylation of AktSer473 was detected and adding HASF did not change the level of total Akt protein either (FIG. 4A). This transient phosphorylation and activation of Akt in cardiomyocytes was almost completely blocked by pre-incubation with 10 μM of PI3K inhibitor LY2940002 (FIG. 4B). Further analysis of Akt downstream target genes revealed a coincident phosphorylation of GSK3βSer9 at 30 min and 3 h, and a gradually increased phosphorylation of pro-apoptotic BadSer128 at 2-3 h. No effect was observed on either the PI3K negative regulator-PTENSer380 or PDK1Ser241, the traditional kinase that phosphorylates AktThr308 (FIG. 4C).

HASF Secreted from Akt-MSCs Activated Cyclin-Dependent Kinase 7 (CDK7) and CDK7 in Turn Phosphorylated Specifically AktThr308 but not AktSer473 in Rat Adult Cardiomyocytes

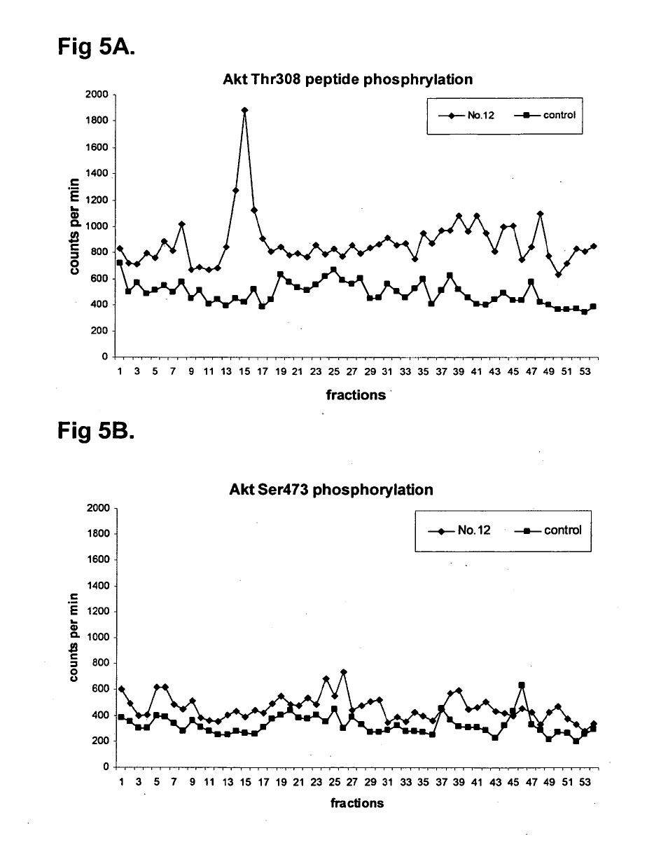

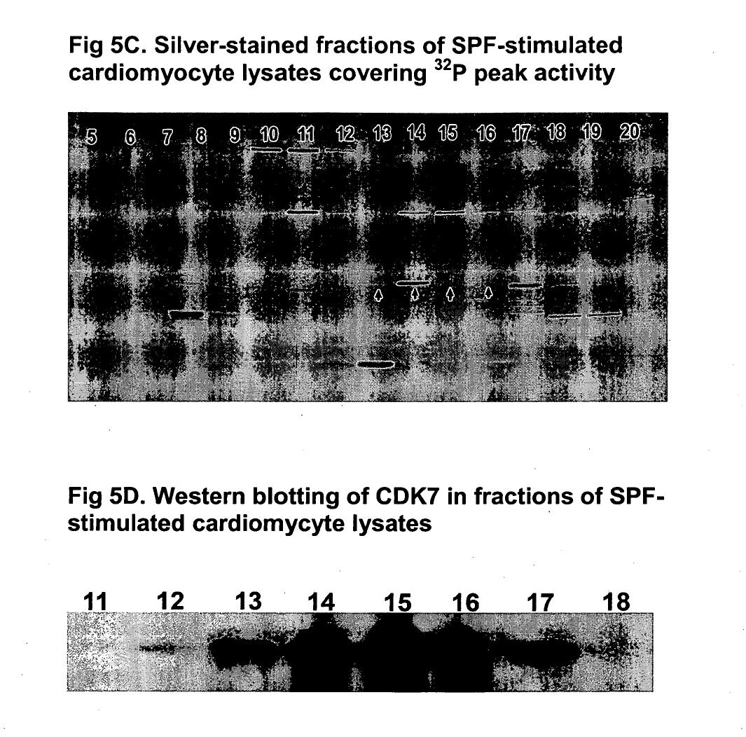

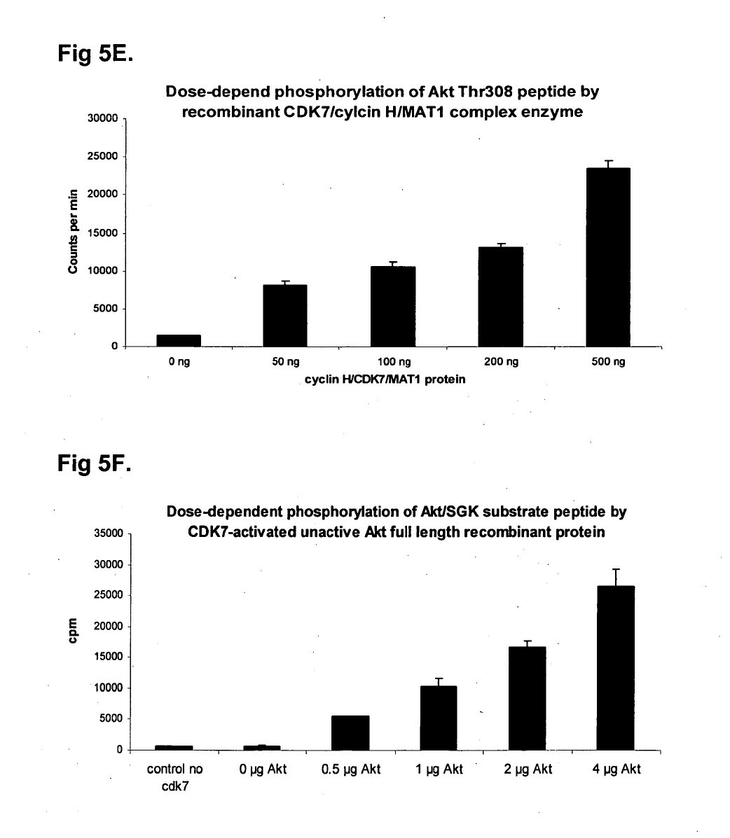

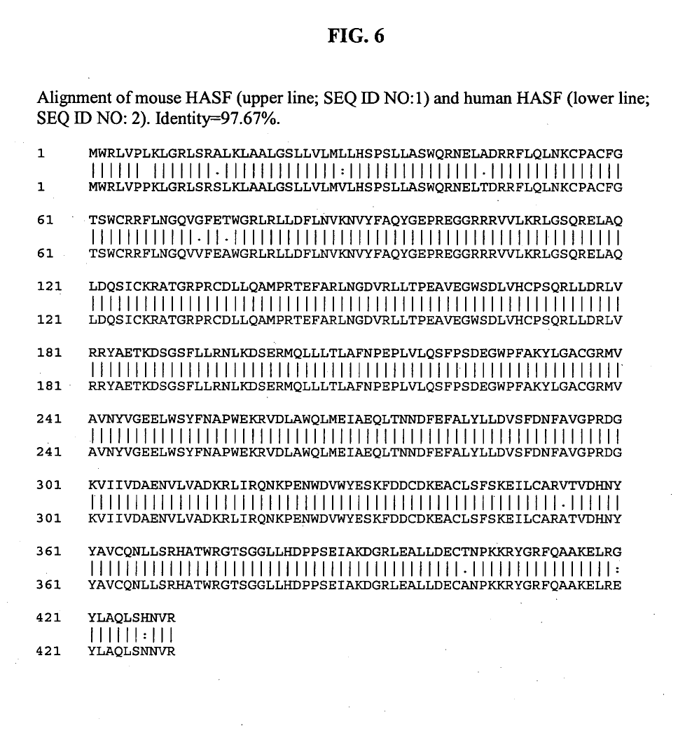

In contrast to the traditional concept that PDK1 is responsible for phosphorylation and activation of both AktThr308 and AktSer473, no changes of the phosphorylation state of PDK1 in cardiomyocytes was observed using different phospho-anti-PDK1 antibodies. A kinase activated by HASF which phosphorylates Akt specifically at Thr308 was identified. As shown in FIGS. 5A and 5B, compared with the vehicle control PBS treated cardiomyocytes, HASF stimulated cardiomyocytes lysates exhibited a peak of 32P radioactive counts among fractions 13-17. This peak is unique only in the lysates of HASF stimulated cardiomyocytes and only in the assays using Akt peptide Thr308, no noticeable peaks observed in Akt peptide Ser437, which was consistent with the data in western blotting using phosphor-Akt antibodies (FIG. 4A.). Cyclin H, which is the regulator for CDK7, was identified by mass-spectrometry sequencing, among fractions 13-16 on a silver-stained SDS-PAGE gel (FIG. 5C) and the kinase CDK7 within fractions 12-17 using CDK7 antibody by western blotting (FIG. 5D), only in the fractions of HASF stimulated cardiomyocyte lysates. To further confirm whether CDK7 can phosphorylate AktThr308, another enzymatic assay was carried out using CDK7/cyclin H/MAT1 recombinant kinase complex. ³²P radioactivity was incorporated into the Akt peptide Thr308 (FIG. 5E), in a dose-dependent manner. To validate if CDK7 can phosphorylate and activate a full-length Akt protein, instead of just a short synthetic peptide, another assay was done using a full-length but unactive Akt recombinant protein. As shown in FIG. 5F, compared with controls of either no CDK7 enzyme or no unactive Akt protein, the unactive Akt was phosphorylated and activated by CDK7 and the activated full-length Akt protein in turn phosphorylated Akt/SGK substrate peptide.

### Regeneration and Repair of Myocardium

Stem cells play a role in endogenous repair and regeneration of tissues in response to injury, and the transplantation of stem cells is used for regenerative therapy. In the cardiovascular field, acute myocardial infarction and stroke are two conditions in which stem cell therapy holds particular promise. However, significant knowledge gaps exist in the understanding of stem cell biology and actions in tissue repair and regeneration. However, a major limitation of stem cell therapy is the poor viability of the transplanted cells in vivo.

Hypoxia activated/phosphorylated Akt was found to increase stem cell survival in vitro and in vivo. MSC with Akt overexpression secreted cytokines that exert paracrine activities on the ischemic myocardium. MSCs injected into myocardium release a cocktail of angiogenic and anti-apoptotic factors, which account for the angiogenic and cytoprotective effects on the injured myocardium. Paracrine factors released from stem cells also activate resident cardiac stem cells for myocardium regeneration.

Protein sequence alignment of human and mouse HASF revealed a ˜98% similarity, indicating a high conservation of this protein between species during evolution. Mouse HASF mRNA is abundantly present in the ovary, brain, liver and embryo, with modest expression in the lung, thymus, spleen and heart, and no expression in the kidney and testes. Transfection of HEK 293 cells with an expression construct harboring full-length human HASF resulted in a prominent accumulation of this protein in the culture media as detected by western blotting suggesting that HASF is a secreted protein. Bioinformatics via online predictions indicate that HASF possesses a typically N-signal peptide and without any hydrophobic transmembrane domains as seen in most classical secreted proteins.

HASF is a ˜40 KDa protein with 10 cysteines in total and this cysteine-rich structure made purification difficult. HASF has impressive cellular prosurvival activity. It protected cardiomyocytes against apoptotsis both in vitro and in vivo. Using the purified recombinant protein, we observed that HASF protected H9C2 myocytes against H₂O₂ induced early apoptosis (˜50%) by Annexin V/PI staining. In this assay, HASF has activity comparable to IGF1 and appears to be a growth factor by a protein function prediction. The recombinant protein at a final concentration of 10 nM dramatically inhibited Caspase 9 and Caspase 3/7 activities in H₂O₂ induced apoptosis in rat adult cardiomyocytes at various time points and prevented DNA fragmentation to a noticeable extend in the late stage apoptosis as well. The release of Cytochrome C from mitochondria into cytosolic compartment was greatly reduced by pre-incubation of 10 nM of HASF with rat adult cardiomyocytes challenged with 100 M of H₂O₂. HASF also maintained Bcl-2 protein level on mitochondria during H2O2 induced apoptosis but did not prevent the translocation of Bax protein from cytosol onto mitochondria. Importantly, intramyocardium injection of 1 μg of HASF into rat heart ischemia/reperfusion model significantly protected in vivo apoptosis analyzed by TUNEL staining (˜69% reduction) and TTC staining, leading to a 60% dramatic reduction of myocardial infarction compared with PBS injected animals. Hearts received 1 μg of ASF injection exhibited much less fibrosis (˜61% reduction) as evidenced by Masson's Accustain Trichrome staining for collagen as well 2 and 4 weeks later.

HASF binds a receptor kinase and/or cell surface receptor, to deliver a survival signal into the cardiomyocytes and activate anti-apoptosis pathway(s), accounting for the significant cardio-protection observed previously in Akt-MSCs. Pre-incubation of cardiomyocytes with 10 nM of HASF transiently activated PI3K-Akt pathway in cardiomyocytes, in which AktThr308 but not AktSer473 was phosphorylated, peaking at 30 min, decreased slightly afterwards and then increase up again at 3 h time point, with a coincident phosphorylation of Akt downstream substrates like GSK3βSer9 at 30 min and 3 h, and a gradually increased phosphorylation of pro-apoptotic BadSer128 at 2-3 h respectively. No effect could be observed on PTENSer380, the negative regulator of PI3K-Akt pathway; and on PDK1, the kinase that phosphorylates Akt at both Thr208 and Ser47372.

Studies were undertaken to further investigate the nature of the HASF activated kinase upstream of Akt. With the in vitro kinase assays and HPLC isolation, the data strongly indicate that CDK7 is responsible for the phosphorylation of AktThr308. This discovery further establishes a novel signaling pathway of HASF involving the activation of CDK7 and phosphorylation of AktThr308 and its downstream targets including the inactivation of pro-apoptotic initiator Caspase 9 and subsequent effector Caspase 3/7, phosphorylation/inactivation of GSK3β to reduce apoptosis and enhance survival, phosphorylation/inactivation of pro-apoptotic Bad, stabilize mitochondrial Bc-2 and prevent Cytochrome C release and etc, all of which directly accounts for the dramatic prosurvival effect in vitro and in vivo observed.

HASF exerts its anti-apoptosis function through the transient activation of CDK7 and subsequent phosphorylation of Akt pathways in adult rat cardiomyocytes. HASF released by Akt MSC has an autocrine effect on the stem cell itself. Akt MSC in response to hypoxia express and release HASF which subsequently activate Akt in MSC to provide a positive feedback loop thereby increasing stem cell viability and further amplifying Akt paracrine effects including the release of sfrp2 that enhance target tissue cell survival, repair and regeneration. HASF can influence stem cell proliferation/differentiation as well rendering adult cardiomyocytes to re-enter cell cycle and participate in tissue regeneration.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing cell death, comprising contacting an injured or diseased tissue with a composition comprising a purified Hypoxia regulated Akt MSC Paracrine Secreted Factor (HASF).

2. The method of claim 1, wherein said tissue is cardiac tissue.

3. The method of claim 1, wherein said tissue is a myocardium.

4. The method of claim 1, wherein said tissue is selected from the group consisting of cardiac tissue, liver tissue, kidney tissue, or neurological tissue.

5. The method of claim 1, wherein said HASF comprises the amino acid sequence of SEQ ID NO:1 or 2.

6. A method for reducing myocardial infarct size, comprising administering to an individual suffering from or having suffered from a myocardial infarction, a composition comprising purified HASF.

7. The method of claim 6, further comprising administering a second therapeutic agent.

8. The method of claim 7, wherein said second therapeutic agent is selected from the group consisting of an anti-apoptotic agent, a protein kinase C (PKC) modulator, and an anti-thrombotic agent.

9. The method of claim 6, wherein said HASF is administered by direct injection into myocardium.

10. The method of claim 6, wherein said HASF is administered by infusion into a coronary artery.

11. Use of HASF in the manufacture of a medicament to reduce cell death in an ischemic tissue.

12. Use of HASF in the manufacture of a medicament for preserving or storing a tissue or organ ex vivo.
